(19) United States
(12) Patent Application Publication
(10) Pub. No.: US 2019/0233503 A1
(43) Pub. Date: Aug. 1, 2019

(54) METHOD OF MANUFACTURING PROTHROMBIN COMPLEX CONCENTRATE FROM FRACTION III AND NON-PROTHROMBIN COMPLEX CONCENTRATE FROM FRACTION IV

(71) Applicant: Kieu Hoang, Westlake Village, CA (US)

(72) Inventor: Kieu Hoang, Westlake Village, CA (US)

(21) Appl. No.: 16/379,559

(22) Filed: Apr. 9, 2019

Related U.S. Application Data
(62) Division of application No. 15/278,729, filed on Sep. 28, 2016.
(60) Provisional application No. 62/237,619, filed on Oct. 6, 2015.

(51) Int. Cl. C07K 16/06; C07K 1/18; C07K 1/34; C07K 1/14; C07K 16/10; C07K 1/36

(52) U.S. Cl. CPC C07K 16/065; C07K 1/18; C07K 1/34; A61K 2039/54; C07K 16/109; C07K 1/36; C07K 1/145

(57) ABSTRACT

The present subject matter is directed to a method of manufacturing and purifying an intravenous injection of prothrombin complex concentration (PCC) from plasma Fraction III and a method of manufacturing and purifying an intravenous injection of non-PCC from plasma Fraction IV. The intravenous injection of PCC and non-PCC obtained from the method can be administered to a patient in need thereof for stopping replication, killing and preventing HIV-1 and HIV-2 in a patient.

### CROSS-REFERENCE

The present application is a divisional application of U.S. patent application Ser. No. 15,278,729, which was filed on Sep. 28, 2016, which application claims the benefit of U.S. Provisional Patent Application 62/237,619, which was filed on Oct. 6, 2015. Each of these applications was incorporated herein by reference.

### TECHNICAL FIELD

The present subject matter relates to an intravenous injection of Prothrombin Complex Concentrate (PCC) manufactured from plasma Fraction III for Hemophilia B or Hemophilia A patients. The present subject matter also relates to an intravenous injection of non-PCC manufactured from plasma Fraction IV for non-hemophilic patients. In particular, the present subject matter is associated with methods of using an intravenous injection of the manufactured PCC for curing and preventing Hemophilia A and Hemophilia B patients infected with viruses HIV-1 and HIV-2, and methods of using an intravenous injection of the manufactured non-PCC for curing and preventing non-hemophilic patients infected with viruses HIV-1 and HIV-2.

### BACKGROUND

Prothrombin complex concentrate (PCC) is a combination of human coagulation factors II, VII, IX, and X, usually prepared from human blood plasma. In clinical practice, PCC is administered to reach quick homeostasis, such as for bleeding episodes or Hemophilia A patients with factor VIII inhibitors. PCC may be used to reverse the effect of warfarin and other anti-coagulants and may be used when such a patient must undergo an emergency operation treatment.

Hemophilia A is known as classic hemophilia or factor VIII deficiency and is a genetic disorder. Hemophilia A occurs when factor VIII, a clotting protein, is missing or defective. Hemophilia B is a genetic disorder where the clotting protein factor IX is missing or defective and is less common than Hemophilia A.

### SUMMARY

There are 19 existing and unknown new found proteins in this PCC formulation, among which eight are newly-found proteins KH 11, KH 12, KH 13, KH 14, KH 15, KH 16, KH 17, and KH 18.

According to the present subject matter, PCC may now be produced from plasma Fraction III paste, which includes eight newly-found proteins for intravenous injection to cure and to prevent HIV-1 and HIV-2.

According to another embodiment of the present subject matter, non-PCC may now be produced from plasma Fraction IV paste for non-hemophilic patients to cure and to prevent HIV-1 and HIV-2.

An embodiment of the present subject matter is directed to the 19 existing and newly-found proteins present in the PCC extracted from Fraction III of human plasma. Of those, eight are newly-found proteins (KH 11, KH 12, KH 13, KH 14, KH 15, KH 16, KH 17, and KH 18) and 11 are existing proteins, which are processed and purified to make a product of PCC. With the addition of newly-found proteins, the intravenous solution of PCC not only stops replication of HIV-1 and HIV-2, but also prevents HIV-1 and HIV-2 virus infections.

An embodiment of the present subject matter is directed to a method of manufacturing and purifying an intravenous injection of PCC from plasma Fraction III comprising the steps:
a) reconstituting a Fraction III paste in a buffer to create a Fraction III suspension;
b) adjusting pH and temperature of the Fraction III suspension;
c) performing PEG precipitation of the Fraction III suspension;
d) centrifuging the Fraction III suspension and collecting a supernatant;
e) filtering the supernatant with a 10CP+90SP filter;
f) performing solvent detergent virus inactivation of the supernatant;
g) undergoing weak anion exchange chromatography of the supernatant;
h) twice washing the supernatant and eluting two to three times;
i) ultra-filtering the supernatant with a 10K membrane;
j) adjusting pH of the supernatant;
k) adjusting activity of a human factor IX of the supernatant;
l) performing aseptic filtration and nano filtration of the supernatant for virus removal; and
m) filling and lyophilizing the supernatant to obtain the intravenous injection of PCC.

An embodiment of the present subject matter is directed to a method of manufacturing and purifying an intravenous injection of non-PCC from plasma Fraction IV comprising the steps:
a) dissolving a Fraction IV paste (P1) and mixing for 3-4 hours;
b) filtering the paste (P1) and collecting supernatant (S1);
c) adding A50 gum to the supernatant (S1) and mixing for one hour;
d) filtering the supernatant (S1) and collecting a second paste (P2);
e) adding A50 wash buffer to the second paste (P2) and eluting the buffer;
f) collecting a second supernatant (S2) using a 0.45 filter;
g) adding a solvent detergent to the second supernatant (S2);
h) diluting the second supernatant (S2) by adding A50 gum;
i) filtering and collecting a third paste (P3);
j) adding A50 wash buffer to the third paste (P3) and eluting the buffer;
k) collecting a third supernatant (S3) using a 0.45 filter;
k) ultra-filtering the third supernatant (S3) and adjusting pH; and
m) filling and lyophilizing the supernatant to obtain the intravenous injection of non-PCC.

An embodiment of the present subject matter is directed to an intravenous injection of PCC produced according to the method of manufacturing and purifying an intravenous injection of PCC from Fraction III.

An embodiment of the present subject matter is directed to an intravenous injection of non-PCC produced according to the method of manufacturing and purifying an intravenous injection of non-PCC from Fraction IV.

An embodiment of the present subject matter is directed to a method of treatment for a patient comprising administering the intravenous injection of PCC obtained from the method of manufacturing and purifying an intravenous injection of PCC from Fraction III to a patient in need thereof, wherein the intravenous injection of PCC transforms or repairs damaged and sick cells to become healthy cells, wherein the intravenous injection of PCC protects cellular alterations, and wherein the intravenous injection of PCC sends signals to the patient's body to produce new cells that are healthy, thereby preventing the new cells from being affected by intracellular and extracellular damaging signals.

An embodiment of the present subject matter is directed to a method of treatment for a patient comprising administering the intravenous injection of non-PCC obtained from the method of manufacturing and purifying an intravenous injection of non-PCC from Fraction IV to a patient in need thereof, wherein the intravenous injection of non-PCC transforms or repairs damaged and sick cells to become healthy cells, wherein the intravenous injection of non-PCC protects cellular alterations, and wherein the intravenous injection of non-PCC sends signals to the patient's body to produce new cells that are healthy, thereby preventing the new cells from being affected by intracellular and extracellular damaging signals.

An embodiment of the present subject matter is directed to a method of stopping replication of HIV-1 and HIV-2 in a patient comprising administering the intravenous injection of PCC obtained from the method of manufacturing and purifying an intravenous injection of PCC from Fraction III, or non-PCC obtained from the method of manufacturing and purifying an intravenous injection of non-PCC from Fraction IV, to a patient in need thereof.

An embodiment of the present subject matter is directed to a method of killing HIV-1 and HIV-2 in a patient comprising administering the intravenous injection of PCC obtained from the method of manufacturing and purifying an intravenous injection of PCC from Fraction III, or non-PCC obtained from the method of manufacturing and purifying an intravenous injection of non-PCC from Fraction IV, to a patient in need thereof.

An embodiment of the present subject matter is directed to a method of preventing infection of HIV-1 and HIV-2 in a patient comprising administering the intravenous injection of PCC obtained from the method of manufacturing and purifying an intravenous injection of PCC from Fraction III, or non-PCC obtained from the method of manufacturing and purifying an intravenous injection of non-PCC from Fraction IV, to a patient in need thereof.

An embodiment of the present subject matter is directed to a method of curing and preventing Hemophilia A in a patient comprising administering the intravenous injection of PCC obtained from the method of manufacturing and purifying an intravenous injection of PCC from Fraction III to a patient in need thereof.

### BRIEF DESCRIPTION OF THE DRAWINGS

FIG. 1 is a flow chart depicting the production of PCC from Fraction III.

FIG. 2 is a flow chart depicting the production of non-PCC from Fraction IV.

FIG. 3 shows 2D electrophoresis of PCC from Fraction III, which shows newly-found proteins KH 11, KH 12, KH 13, KH 14, KH 15, KH 16, KH 17, and KH 18.

FIG. 4 shows an SDS-PAGE of the PCC plasma from Fraction III and non-PCC from Fraction IV.

FIG. 5 shows the inhibition rate of AFCC RAAS in 5 HIV-1 strains and the control virus AMYL. Results show the inhibition rate is about 60% when the dilution is less than 1:40. Inhibition is also observed in the control virus AMLV. Cell toxicity is found in high concentrations via observation of cell morphology 48 hours after treatment.

FIG. 6 shows the results of a cell toxicity test of AFCC RAAS. Test samples were diluted at 1:1.5 to start and then 1:4.5, 1:13.5, 1:40.5, 1:121.5, 1:364.5, 1:1093.5, and 1:3280.5, where the dilution was a three-fold dilution with eight dilutions in total. The test kit used was cell counting kit 8 (CCK-8), where the procedure was performed according to the manufacturer's manual. Results show some cell toxicity of RAAS, which likely causes the inhibition of HIV.

### DETAILED DESCRIPTION

Unless defined otherwise all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language; however, it will be understood by one of skill in the art, that in some specific instances, an embodiment can alternatively be described using the language “consisting essentially of” or “consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

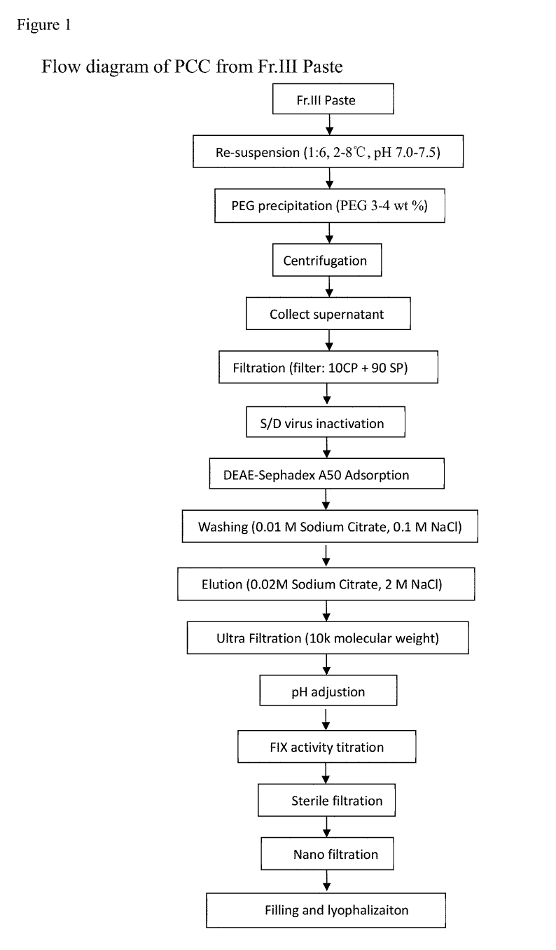

As shown in FIG. 1, an embodiment of the present subject matter is directed to a method of manufacturing and purifying an intravenous injection of PCC from plasma Fraction III comprising the steps:
a) reconstituting a Fraction III paste in a buffer to create a Fraction III suspension;
b) adjusting pH and temperature of the Fraction III suspension;
c) performing PEG precipitation of the Fraction III suspension;
d) centrifuging the Fraction III suspension and collecting a supernatant;
e) filtering the supernatant with a 10CP+90SP filter;
f) performing solvent detergent virus inactivation of the supernatant;
g) undergoing weak anion exchange chromatography of the supernatant;
h) twice washing the supernatant and eluting two to three times;
i) ultra-filtering the supernatant with a 10K membrane;
j) adjusting pH of the supernatant;
k) adjusting activity of a human factor IX of the supernatant;
l) performing aseptic filtration and nano filtration of the supernatant for virus removal; and
m) filling and lyophilizing the supernatant to obtain the intravenous injection of PCC.

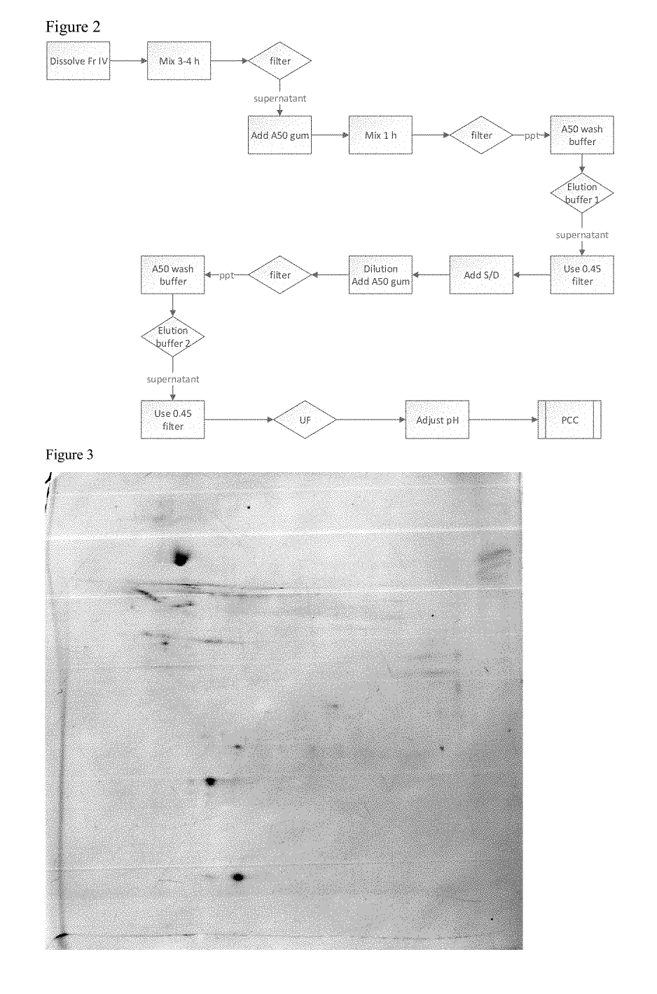

As shown in FIG. 2, another embodiment of the present subject matter is directed to a method of manufacturing and purifying an intravenous injection of non-PCC from plasma Fraction IV comprising the steps:
a) dissolving a Fraction IV paste (P1) and mixing for 3-4 hours;
b) filtering the paste (P1) and collecting supernatant (S1);
c) adding A50 gum to the supernatant (S1) and mixing for one hour;
d) filtering the supernatant (S1) and collecting a second paste (P2);
e) adding A50 wash buffer to the second paste (P2) and eluting the buffer;
f) collecting a second supernatant (S2) using a 0.45 filter;
g) adding a solvent detergent to the second supernatant (S2);
h) diluting the second supernatant (S2) by adding A50 gum;
i) filtering and collecting a third paste (P3);
j) adding A50 wash buffer to the third paste (P3) and eluting the buffer;
k) collecting a third supernatant (S3) using a 0.45 filter;
l) ultra-filtering the third supernatant (S3) and adjusting pH; and
m) filling and lyophilizing the supernatant to obtain the intravenous injection of non-PCC.

An embodiment of the present subject matter is directed to an intravenous injection of PCC produced according to the method of manufacturing and purifying an intravenous injection of PCC from Fraction III. An embodiment of the present subject matter is directed to purified PCC containing eight newly-found proteins, namely KH 11, KH 12, KH 13, KH 14, KH 15, KH 16, KH 17, and KH 18. In an embodiment, Fraction III is obtained by Cohn ethanol fractionation of plasma and comprises one or more newly-found proteins KH 11, KH 12, KH 13, KH 14, KH 15, KH 16, KH 17, and KH 18.

In an embodiment of the present subject matter, the Fraction III suspension is resuspended in a buffer containing up to 10% heparin and up to 80 mM sodium citrate and pH and temperature are adjusted. In an embodiment of the present subject matter, the Fraction III suspension is precipitated with PEG at a final concentration of 4.0-10.0 wt %. In an embodiment of the present subject matter, the solvent detergent virus inactivation comprises adding TNBP to a final concentration of 0.3% and Tween-80 to a final concentration of 1.0% at 25° C. for 6 hours. In an embodiment of the present subject matter, the weak anion exchange chromatography is DEAE A-50 at a final concentration of 4-10 wt %.

In an embodiment of the present subject matter, washing the supernatant comprises using a washing buffer comprising up to 1.0 M sodium citrate and up to 2.0 M NaCl for two times. In an embodiment of the present subject matter, washing the supernatant comprises using a washing buffer comprising up to 2.0 M sodium citrate and up to 2.0 M NaCl for two to three times. In an embodiment of the present subject matter, the aseptic filtration is at 0.22 μm. In an embodiment of the present subject matter, the nano filtration is at 20 nm.

An embodiment of the present subject matter is directed to a method of treatment for a patient comprising administering the intravenous injection of PCC obtained from the method of manufacturing and purifying an intravenous injection of PCC from Fraction III to a patient in need thereof, wherein the intravenous injection of PCC transforms or repairs damaged and sick cells to become healthy cells, wherein the intravenous injection of PCC protects cellular alterations, and wherein the intravenous injection of PCC sends signals to the patient's body to produce new cells that are healthy, thereby preventing the new cells from being affected by intracellular and extracellular damaging signals.

An embodiment of the present subject matter is directed to a method of treatment for a patient comprising administering the intravenous injection of non-PCC obtained from the method of manufacturing and purifying an intravenous injection of non-PCC from Fraction IV to a non-hemophilic patient in need thereof, wherein the intravenous injection of non-PCC transforms or repairs damaged and sick cells to become healthy cells, wherein the intravenous injection of non-PCC protects cellular alterations, and wherein the intravenous injection of non-PCC sends signals to the patient's body to produce new cells that are healthy, thereby preventing the new cells from being affected by intracellular and extracellular damaging signals.

An embodiment of the present subject matter is directed to a method of stopping replication of HIV-1 and HIV-2 in a patient comprising administering the intravenous injection of PCC obtained from the method of manufacturing and purifying an intravenous injection of PCC from Fraction III to a patient in need thereof. In an embodiment, the patient is a Hemophilia B patient.

An embodiment of the present subject matter is directed to a method of stopping replication of HIV-1 and HIV-2 in a patient comprising administering the intravenous injection of non-PCC obtained from the method of manufacturing and purifying an intravenous injection of non-PCC from Fraction IV to a patient in need thereof. In an embodiment, the patient is a non-hemophilic patient.

An embodiment of the present subject matter is directed to a method of killing HIV-1 and HIV-2 in a patient comprising administering the intravenous injection of PCC obtained from the method of manufacturing and purifying an intravenous injection of PCC from Fraction III to a patient in need thereof. In an embodiment, the patient is a Hemophilia B patient.

An embodiment of the present subject matter is directed to a method of killing HIV-1 and HIV-2 in a patient comprising administering the intravenous injection of non-PCC obtained from the method of manufacturing and purifying an intravenous injection of non-PCC from Page 11. Fraction IV to a patient in need thereof. In an embodiment, the patient is a non-hemophilic patient.

An embodiment of the present subject matter is directed to a method of preventing infection of HIV-1 and HIV-2 in a patient comprising administering the intravenous injection of PCC obtained from the method of manufacturing and purifying an intravenous injection of PCC from Fraction III to a patient in need thereof. In an embodiment, the patient is a Hemophilia B patient.

An embodiment of the present subject matter is directed to a method of preventing and killing HIV-1 and HIV-2 in a patient comprising administering the intravenous injection of non-PCC obtained from the method of manufacturing and purifying an intravenous injection of non-PCC from Fraction IV to a patient in need thereof. In an embodiment, the patient is a non-hemophilic patient.

An embodiment of the present subject matter is directed to a method of curing and preventing Hemophilia A with inhibitors in a patient comprising administering the intravenous injection of PCC obtained from the method of manufacturing and purifying an intravenous injection of PCC from Fraction III to a patient in need thereof.

In an embodiment, any of these or any combination of the eight newly-found proteins has the ability to stop replication of HIV-1 and HIV-2. In an embodiment, any of these or any combination of the eight newly-found proteins has the ability to kill HIV-1 and HIV-2. In an embodiment, any of these or any combination of the eight newly-found proteins has the ability to prevent infection of HIV-1 and HIV-2. In an embodiment, any of these or any combination of the newly-found proteins has the following abilities: 1) transform/repair DAMAGED and SICK cells to become GOOD healthy cells, 2) protect cellular alterations, and 3) signal the body to produce new, healthy cells immunized from intracellular and extracellular damaging signals. In an embodiment, any of these or any combination of the 19 proteins in PCC from Fraction III (Fr. III) has the ability to stop replication of HIV-1 and HIV-2, kill HIV-1 and HIV-2, and prevent infection of HIV-1 and HIV-2.

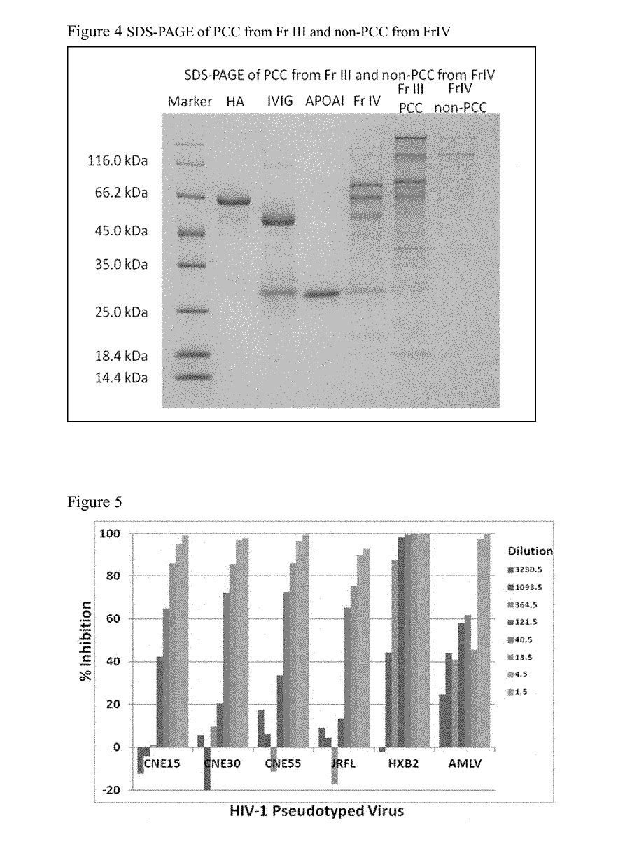

As shown in FIG. 4, the method of manufacturing and purifying an intravenous injection of PCC from plasma Fraction III and the method of manufacturing and purifying an intravenous injection of non-PCC from plasma Fraction IV each provide an improved method for manufacturing and purifying PCC and non-PCC, respectively. In this respect, sodium dodecyl sulphate-polyacrylamide gel electrophoresis (SDS-PAGE) was used in order to provide the results of manufacturing and purifying PCC and non-PCC from Fraction III and IV, respectively. The SDS-PAGE results provide the average molecular weight of both the PCC and non-PCC from Fractions III and IV, respectively.

### EXAMPLES

### In Vitro Testing

The intravenous injection of PCC with the eight newly-found proteins was tested by the Comprehensive National AIDS Research Center, Tsinghua University in China, which concluded that PCC having the code name AFCC RAAS has the ability to stop replication of and kill the HIV virus. The supplementary results of the neutralization of HIV-1 Env-pseudotyped virus follow.

### Samples and Control

The test sample used was AFCC RAAS.

Five strains of the virus were tested. In particular, the strains tested include BC recombinant subtype viruses CNE15 and CNE30, CRF01_AE recombinant subtype virus CNE55, and standard HIV-1 virus strains HXB2 and JRFL. All of the HIV-1 virus strains have CCR5 receptor affinity, with the exception of HXB2, which has CXCR4 receptor affinity.

The control virus used was AMLV.

### Test Method

The test samples were diluted at 1:1.5 to start and then at 1:4.5, 1:13.5, 1:40.5, 1:121.5, 1:364.5, 1:1093.5, and 1:3280.5. The dilution was a three-fold dilution with eight dilutions in total.

### Results

FIG. 5 shows the inhibition rate of AFCC RAAS in the five HIV-1 strains and the control virus AMLV. Results indicate the inhibition rate is about 60% when the dilution was less than 1:40 and inhibition also was observed in the control virus AMLV. Cell toxicity was found in high concentrations via observing cell morphology 48 hours after treatment. The cell toxicity test was then conducted.

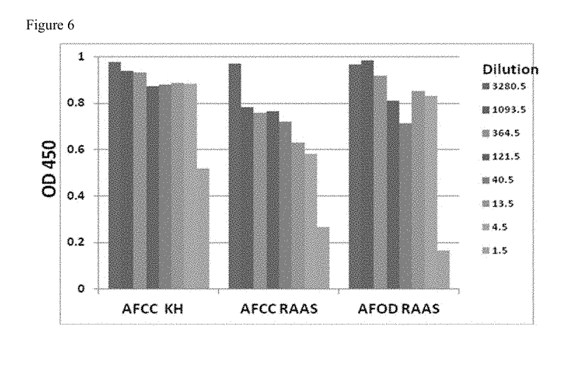

FIG. 6 shows the results of the cell toxicity test. In particular, the toxicity of AFCCKH, AFOD RAAS 101, and AFCC RAAS were tested. Test samples were diluted at 1:1.5 to start and then at 1:4.5, 1:13.5, 1:40.5, 1:121.5, 1:364.5, 1:1093.5, and 1:3280.5. The dilution was a three-fold dilution with eight dilutions in total. The test kit was cell counting kit 8 (CCK-8), and the procedure was carried out according to manufacturer's manual. Results indicate there is some cell toxicity of RAAS. Thus, inhibition of HIV virus is likely caused by cell toxicity.

To decrease the toxicyte to cell, and ensure the high inhibition of virus at high protein concentration, the protein concentration may be further increased. Furthermore, the cell culture medium, DMEM+10% FBS, may be used as the diluent of products when preparing the samples.

With the information contained herein, various departures from precise descriptions of the present subject matter will be readily apparent to those skilled in the art to which the present subject matter pertains, without departing from the spirit and the scope of the below claims. The present subject matter is not considered limited in scope to the procedures, properties, or components defined, since the preferred embodiments and other descriptions are intended only to be illustrative of particular aspects of the presently provided subject matter. Indeed, various modifications of the described modes for carrying out the present subject matter which are obvious to those skilled in chemistry, biochemistry, or related fields are intended to be within the scope of the following claims.

I claim:

1. A method of manufacturing and purifying an intravenous injection of prothrombin complex concentration (PCC) from plasma Fraction III, comprising the steps:
a) reconstituting a Fraction III paste in a buffer to create a Fraction III suspension;
b) adjusting pH and temperature of the Fraction III suspension;
c) performing PEG precipitation of the Fraction III suspension;
d) centrifuging the Fraction III suspension and collecting a supernatant;
e) filtering the supernatant with a 10CP+90SP filter;
f) performing solvent detergent virus inactivation of the supernatant;
g) undergoing weak anion exchange chromatography of the supernatant;
h) twice washing the supernatant and eluting two to three times;
i) ultra-filtering the supernatant with a 10K membrane;
j) adjusting pH of the supernatant;
k) adjusting activity of a human factor IX of the supernatant;
l) performing aseptic filtration and nano filtration of the supernatant for virus removal; and
m) filling and lyophilizing the supernatant to obtain the intravenous injection of PCC.

2. The method of claim 1, wherein said plasma Fraction III is obtained by Cohn ethanol fractionation of plasma and comprises newly-found proteins KH 11, KH 12, KH 13, KH 14, KH 15, KH 16, KH 17, and KH 18.

3. The method of claim 1, wherein the Fraction III suspension is re-suspended in a buffer containing up to 10% heparin and up to 80 mM sodium citrate and pH and temperature are adjusted.

4. The method of claim 1, wherein the Fraction III suspension is precipitated with PEG at a final concentration of 4.0-10.0 wt %.

5. The method of claim 1, wherein the solvent detergent virus inactivation comprises adding TNBP to a final concentration of 0.3% and Tween-80 to a final concentration of 1.0% at 25° C. for 6 hours.

6. The method of claim 1, wherein the weak anion exchange chromatography is conducted using DEAE A-50 at a final concentration of 4-10 wt %.

7. The method of claim 1, wherein washing the supernatant comprises using a washing buffer comprising up to 1.0 M sodium citrate and up to 2.0 M NaCl for two times.

8. The method of claim 1, wherein washing the supernatant comprises using a washing buffer comprising up to 2.0 M sodium citrate and up to 2.0 M NaCl for two to three times.

9. The method of claim 1, wherein the aseptic filtration is at 0.22 μm.

10. The method of claim 1, wherein the nano filtration is at 20 nm.

11. A method of treatment for a patient comprising administering the intravenous injection of PCC obtained from the method of claim 1 to a patient in need thereof,
wherein the intravenous injection of PCC transforms or repairs damaged and sick cells to become healthy cells,
wherein the intravenous injection of PCC protects cellular alterations, and
wherein the intravenous injection of PCC sends signals to the patient's body to produce new cells that are healthy, thereby preventing the new cells from being affected by intracellular and extracellular damaging signals.

12. A method of stopping replication of HIV-1 and HIV-2 in a patient comprising administering the intravenous injection of PCC obtained from the method of claim 1 to a patient in need thereof.

13. The method of claim 12, wherein the patient in need thereof is a Hemophilia B patient.
